(19) United States
(12) Patent Application Publication
(10) Pub. No.: US 2007/0149767 A1
(43) Pub. Date: Jun. 28, 2007

(54) Agent

(76) Inventors: Michael Gaston Joseph Grandgeorge, Lyon (FR); Darrell Sleep, West Bridgford (GB)

Correspondence Address: SYNNESTVEDT & LECHNER, LLP, 2600 ARAMARK TOWER, 1101 MARKET STREET, PHILADELPHIA, PA, 191072950, US

(21) Appl. No.: 10/548,915

(22) PCT Filed: Mar. 15, 2004
(86) PCT No.: PCT/GB04/01100

§ 371 Date: Jul. 5, 2006

(30) Foreign Application Priority Data
Mar. 15, 2003 (GB) 03059896

(51) Int. Cl. C07K 14/765

(52) U.S. Cl. 530/363; 514/12

(57) ABSTRACT

An agent having a greater half-life than naturally produced albumin in a patient with NS, the agent comprising an albumin-like first polypeptide bound to a second polypeptide, wherein the second polypeptide, when bound to the albumin-like first polypeptide is therapeutically inert and wherein if the agent consists of two albumin molecules, then they are covalently joined to one another other than solely by means of one or more cysteine-cysteine disulphide bridges.

The present invention relates to an agent for treatment of renal disorders and associated conditions, methods and means for producing the agent and uses of the agent in treatment of renal disorders and associated conditions. More particularly the present invention relates to modified albumin having a lower rate of excretion from the blood of a patient with nephrotic syndrome than naturally produced albumin.

Nephrosis is a disease of the kidneys characterised by hypoalbuminemia, hypercholesterolemia and oedema. Nephrotic syndrome (NS) is marked by severe leakiness of the glomerular filtration system, resulting in massive losses of plasma proteins (Peters, 1996, All About Albumin: Biochemistry, Genetics and Medical Applications, Academic Press, Inc., San Diego, Calif.).

It has been shown that a normal glomerular barrier prevents albuminuri a (the presence of albumin in the urine) by a combination of charge- and size-selective properties and that impairment of both of these properties is required to account for observable levels of proteinuria in the nephrotic subjects studied (Blouch et al, 1997, Am J. Physiol., 273(3 Pt 2): F430-437). Proteins under 78 kDa in size are particularly susceptible to proteinuria in patients with NS (Peters, 1996, op. cit.)

Human serum albumin (HSA) is typically about 66 kDa in size. In patients with NS the daily loss of albumin typically increases from about 10 mg to 1-8 g, although it can be as high as 17 g (Peters, 1996, op. cit.). An incidental finding in NS is a high proportion of albumin S—S-bonded dimers in urine samples that have been stored frozen (Birtwhistle and Hardwicke, 1985, Clin. Chim. Acta, 151, 41-48). Increased catabolism within the renal tubules (Kaysen and Bander, 1990, Am. J. Nephrol., 10 (Suppl. 1), 36-42) adds to the albumin loss, so that the half-life of albumin in the plasma drops from 19 days to about 4 days (Jensen et al, 1967, Clin. Sci., 33, 445-457).

Low serum albumin levels can lead to increased blood lipid content. Hypoalbuminemia has been shown to be an important trigger factor in the elevation of serum lipoprotein (a) levels, which can promote atherosclerotic cardiovascular disease, particularly in patients with NS (Yang et al, 1997, Am. J. Kidney Dis., 30(4):507-513). Since hyperlipidemia is characteristic of NS and parallels the amount of albumin lost, it has been suggested that the paucity of albumin as an acceptor of long chain fatty acids from lipoprotein lipase action may play a role in hyperlipidemia (Peters, 1996, op. cit.).

Typically 30-50% of patients respond by increased albumin synthesis, usually by 30-40% from a normal rate of 13-14 g/day, although increases can be as high as 27 g/day (Peters, 1996, op. cit). In keeping with the dependence of albumin synthesis on the amino acid supply, a high protein intake has been suggested as a treatment for NS (Peters, 1996, op. cit.). Conversely, a low protein diet has also been considered in the treatment of NS (Palmer, 1993, Am J Med Sci, 306(1): 53-67; Kaysen, 1998, Miner. Electrolyte Metab., 24: 55-63) and was recently demonstrated to reduce proteinuria and increase serum albumin levels in nephrotic patients (Giordano et al, 2001, Kidney Int., 60(1): 235-242).

Other treatments have also been proposed for NS. Albumin infusions have been shown to increase plasma volume in severely nephrotic patients but the effect is of short duration, the albumin being immediately eliminated renally (Kuhn et al, 1998, Kidney Int. Suppl., 64: S50-53), whereas a coadministration of albumin and the loop diuretic, furosemide, have been demonstrated to be more effective in the treatment of nephrotic oedema (Bircan et al, 2001, Pediatr. Nephrol., 16(6):497-499). It was concluded that coadministration of human albumin potentiates the action of furosemide in patients with NS, but only modestly (Fliser et al, 1999, Kidney Int., 55(2): 629-634). Furthermore, it has beers shown that recombinant HSA (rHSA) can be used as an equivalent to for HSA prepared from human plasma in therapy with furosemide-resistant NS (Tsukada et al, 1998, Gen. Pharmacol., 31(2): 209-214).

Other suggested therapies include the use of angiotensin-converting enzyme (ACE) inhibitors to reduce protein uria in NS (Bakris, 1993, Postgrad. Med., 93(5): 89-100), prednisolone, cyclophosphamide, chlorambucil and hydrochlorothiazide for the treatment of primary glomerular diseases associated with NS (Kuhn et al, 1998, Kidney Int. Suppl., 64: S50-53) and the treatment of nephrotic patients who have a serum albumin level of under 20 g/litre by routine anticoagulation (Kuhn et al, 1998, op. cit.). It has also been suggested that extensive hyperlipidemia in severe NS can be treated with HMG-CoA reductase inhibitors (Kuhn et al, 1998, op. cit.).

Against this background, it is an object of the present invention to provide an agent for the treatment of renal conditions, such as MS, by providing a modified albumin that has an improved (ie, greater) half-life than naturally produced albumin.

Fusions of albumin are known in the prior art. In fact, albumin is well known to be suitable for use as a carrier of other proteins due to its long half-life and relative lack of biological activity. Thus, the prior art describes albumin fusions with therapeutically active proteins wherein the fusion has the effect of improving the half-life of the therapeutically active proteins, but not the half-life of the albumin. For example, in WO 01/05826, there is provided a chimeric polypeptide containing segments of serum albumin and fragments of biologically active heterologous peptides, the biologically active heterologous peptides being, for example, receptor activators, antigens, or peptides able to interact with a membrane or ion channel and being less than 40% identical to the albumin to which it is fused. Moreover, WO 01/05826 teaches that the chimeric polypeptide provided therein has a half-life that merely approaches, rather than exceeds, the half-life of unfused serum albumin.

U.S. Pat. No. 6,165,470 provides hybrid macromolecules characterised by the fact that they carry either the active domain of a receptor for a virus, or the active domain of a molecule which can bind to a virus, or the active domain of a molecule able to recognise the Fc fragment of immunoglobulins bound to a virus, or the active domain of a molecule able to bind to a ligand that intervenes in a pathologic process, coupled to albumin or a variant of albumin. These hybrids are formed in order to increase the half-life of the active domain.

WO 99/66054 provides erythropoietin analogue-HSA fusion proteins having erythropoietin biological activity. WO 00/69900 provides for the protection of endogenous therapeutic peptides from peptidase activity through conjugation to blood components in order to increase the half-life of the therapeutic peptides. WO 00/69900 prefers to use albumin as a carrier for the therapeutic peptides.

Accordingly, a first aspect of the present invention provides an agent having a greater half-life than naturally produced albumin in a patient with NS, the agent comprising an albumin-like polypeptide bound to a second polypeptide wherein the second polypeptide is therapeutically inert, at least when bound to the albumin-like first polypeptide, and wherein if the agent consists of two albumin molecules, then they are covalently joined to one another other than solely by means of one or more cysteine-cysteine disulphide bridges. In other words, the agent of the first aspect of the invention is not an albumin S—S-bonded dimer as described in Birtwhistle and Hardwicke, 1985 (op. cit).

A second aspect of the present invention provides a composition comprising a pharmaceutically acceptable carrier or diluent and an agent having a greater half-life than naturally produced albumin in a patient with NS, the agent comprising an albumin-like polypeptide bound to a second polypeptide, wherein the second polypeptide, when bound to the albumin-like polypeptide, is therapeutically inert, and wherein when the agent is a dimer of albumin-like polypeptides more than 2% of the albumin-like first polypeptide present in the pharmaceutical composition is bound to a the second polypeptide.

A carrier or diluent is “pharmaceutically acceptable” if it is compatible with an agent of the first or second aspects of the invention and if it is not deleterious to the recipients thereof. Typically, a carrier or diluent will be water or saline which will be sterile and pyrogen free.

When the agent is a dimer of two albumin polypeptides, at least 30%, 40%, 50% or more of the total albumin polypeptide content of the composition of the second aspect of the invention is bound to the second polypeptide. If the agent is not an albumin-albumin dimer, then a lower proportion (such as 3%, 5%, 9%, 10%, 15% or 20%) of the albumin-like first polypeptide may be bound to the second polypeptide, although it is still preferred for at least 30% (etc, as above) to be so bound. Preferably 60%, 70%, 80% or 90%, more preferably 95%, yet more preferably 98%, even more preferably 99%, most preferably substantially 100% of the albumin-like first polypeptide in a pharmaceutical composition of the second aspect of the invention is bound to the second polypeptide.

Preferably, in a composition according to the second aspect of the invention the agent is present as a majority protein component, more preferably as at least 80% of the total protein content of the composition, when determined by Cellulose Acetate Electrophoresis or size excluision chromatography (SEC)-HPLC as described in the Monograph “Albumin solution, human” of Eur. Pharmacopoeia (3rd Ed, 1997-0255), where the band or peak corresponding to the agent represents >80% of the total electrophoreto gram or chromatogram respectively.

An agent according to the first aspect of the invention and an agent used in a composition according to a second aspect of the invention has a greater half-life than naturally produced albumin in a patient with NS. By “greater” with regard to the half-life of the agent in a patient with NS is included the meaning that the agent has a half-life that is at least 1, more preferably at least 3, more preferably at least 5, even more preferably at least 10 days longer than naturally produced albumin in a patient with NS. Typically the half-life of the agent defined in the first and/or second aspect of the invention in a patient with NS is at least twice, three times, five times, ten times, twenty times or more of the half-life of naturally produced albumin in a patient with NS. The half-life of proteins in the body of a patient can be determined by techniques well known in the art. For example, the half life of an agent according to the present invention (“test agent”) may be clinically compared to wild-type, unmodified albumin (“normal” albumin) as follows: Patients with Nephrotic Syndrome are randomly allocated to “normal” albumin or a “test agent”. Each is infused with one i.v. dose of 1.4 g per Kg. This corresponds to a “normal” albumin concentration in plasma of about 35 g/L (the volume of plasma in humans is 40 mL/kg). Plasma concentration of albumin will be measured before administration and repeated every 12 hours to follow the product survival time in the blood circulation. Infusion will be repeated as above as soon as the product concentration decreases under 20 g/L. Albumin measurements can be made using, for example, a cellulose acetate electrophoresis assay as described in the Monograph “Albumin solution, human” of Eur. Pharmacopoeia (3rd Ed, 1997-0255), combined with a total protein assay like the Protein Nitrogen assay described in the same Monograph. The method can be adapted to suit the particular “test agent”. The physiological effects of the agent may also be followed through markers such as blood pressure, cardiac rhythm, or serum lipoprotein (a) levels, to evaluate half-life improvement.

Alternatively, an in vitro test may be used to evaluate half-life. For example, an ultrafiltration membrane set in a stirred dead end ultrafiltration cell or a recirculation ultrafiltration cell, as provided by known laboratory equipment suppliers such as Millipore Corp. may be used. The solutions of “normal” albumin and “test agent” are adjusted to 35 g/L in phosphate buffered saline, pH 7.2 to 7.4, (PBS) to mimic Plasma. Typically, Millipore's standard polysulfone membranes of 10,000 Da, 30,000 Da, 50,000 Da and 100,000 Da molecular weight cut-offs are used. During ultrafiltration of albumin, the concentration of albumin in the starting solution will be compared to that of the ultrafiltrate solution. Ultrafiltration operating conditions are room temperature and transmembrane pressure 0.5 to 2.0 bars. Protein measurements are performed using a protein assay, for example the total protein assay (proteic nitrogen) described in the Monograph “Albumin solution, human” of the Eur. Pharmacopoeia (3rd Ed, 1997-0255). Ulitrafiltration through the 10,000 Da and 30,000 Da cut-off membranes is used as a control, since no difference is expected between “normal” albumin and the “test agent” when run under the same conditions. The concentration of albumin in the ultrafiltrate is expected to be less than about 10% of the starting solution, i.e. <3.5 g/L. Ultrafiltration through a 50,000 Da cut-off membrane will show more than about 10% passage of “normal” albumin and less than about 10% passage of the “test agent”. Ultrafiltration through a 100,000 Da cut-off membrane will show more than 10% passage of “normal” albumin or even more, such as more tlhan 50% passage. The “test agent” will still demonstrate less than about 10% passage through the membrane.

In a preferred embodiment, an agent of the first and/or second aspect of the invention is larger than naturally produced HSA. Typically the agent is from 80 to 250 kDa, more typically 80 to 200 kDa in size preferably at least 100 kDa in size. It may be preferable not to exceed 150 KDa. Around 130 to 135 KDa may be ideal. The molecular weight values are taken to be the ‘average’ molecular weight and are measured using (size exclusion chromatography) SEC-HPLC as described in the Monograph “Albumin solution, human” of Eur. Pharmacopoeia (3rd Ed, 1997-0255). The summit of the main peak is taken to define the average molecular weight. Calibration of the column is performed using a commercially available calibration proteins like ovalbumin (chicken) of molecular weight (MW) 44,000 Da, albumin (bovine serum) of MW 67,000 Da, transferrin of MW 81,000 Da, aldolase (rabbit muscle) of MW 158,000 Da and catalase (bovine liver) of MW 232,000 Da and a SEC-HPLC TSK G4000 SW×1 column (see “Current Protocols in Protein Science”, 2002, John Wiley & Sons Ed), which are relevant for measuring molecular weight between 50,000 Da and 250,000 Da. In case of a test agent having a molecular weight between 65,000 Da and 80,000 Da, molecular weight may be determined instead using the ElectroSpray Mass Spectrometry ES-MS method. For this purpose, the test agent is desalted on reverse-phase high performance liquid chromatography (RP-HPLC) into a volatile solvent. The protein is collected and injected directly into the ElectroSpray Mass Spectrometer, which has been calibrated using Horse Heart Myoglobin. In the spectrometer the test agent molecules are ionised at atmospheric pressure and the resulting ions pass into a quadrupole analyser where the mass to charge ratios are measured. The data is processed to yield a single mass peak indicative of the true molecular weight of the protein. The accuracy of the technique is within 0.01% of the expected value for albumin (i.e. within 7 Da).

An agent of the first and/or second aspect of the invention comprises an albumin-like first polypeptide. Usually the albumin-like polypeptide is albumin. Typically the albumin is human serum albumin (HSA) extracted from serum or recombinant human albumin (rHA) produced by transforming or transfecting an organism or cell line with a nucleotide coding sequence encoding the amino acid sequence of human serum albumin. In this specification, the term “albumin” refers generically to HSA and/or rHA. In one embodiment, the term “albumin-like polypeptide” includes within its meaning variant albumin. A “variant albumin” refers to an albumin protein wherein at one or more positions there have been amino acid insertions, deletions, or substitutions, either conservative or non-conservative, provided that such changes result in an albumin protein for which at least one basic property, for example binding activity (type of and specific activity e.g. binding to bilirubin), osmolarity (oncotic pressure, colloid osmotic pressure), behaviour in a certain pH-range (pH-stability) has not significantly been changed. “Significantly” in this context means that one skilled in the art would say that the properties of the variant may still be different but would not be unobvious over the ones of the original protein.

By “conservative substitutions” is intended combinations such as Gly, Ala; Val, Ile, Leu; Asp, Glu; Asn, Gln; Ser, Thr; Lys, Arg; and Phe, Tyr. Such variants may be made using the methods of protein engineering and site-directed mutagenesis known in the art, such as those disclosed in U.S. Pat. No. 4,302,386 issued 24 Nov. 1981 to Stevens, incorporated herein by reference.

Typically an albumin variant will have more than 40%, usually at least 50%, more typically at least 60%, preferably at least 70%, more preferably at least 80%, yet more preferably at least 90%, even more preferably at least 95%, most preferably at least 98% or more sequence identity with naturally occurring albumin. The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally. The alignment may alternatively be carried out using the Clustal W program. (Thompson et al., 1994). The parameters used may be as follows:

Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

Agents according to the first and second aspects of the invention have a second polypeptide bound to the albumin-like polypeptide. The second polypeptide is a polypeptide that is capable of being bound to the albumin-like first polypeptide as defined above and, when bound, increases the molecular weight (MW) of the albumin-like first polypeptide by an amount that causes the thus bound albumin-like first polypeptide to have a greater half-life than naturally produced albumin in a patient with NS. Typically the second polypeptide has a molecular weight of from 14 to 134 kDa, preferably from 20 to 100 kDa, more preferably from 35 to 66 kDa.

The second polypeptide of the agent of the first and second aspect of the invention is therapeutically inert, at least when bound to the albumin-like first polypeptide. By “therapeutically inert” is meant that the second polypeptide substantially adds no further therapeutically beneficial property to the agent over that provided by albumin (other, of course, than extending its half-life) and is not harmful. By “substantially adds no further therapeutically beneficial property” is included the meaning that the second polypeptide does not have a biologically significant receptor activator activity, antigenicity, or ability to interact with a membrane or ion channel not possessed by albumin. By “substantially adds no further therapeutically beneficial property” is further included the meaning that the second polypeptide cannot act any more effectively than albumin as an active domain of a receptor for a virus, an active domain of a molecule which can bind to a virus, an active domain of a molecule able to recognise the Fc fragment of immunoglobulins bound to a virus, or an active domain of a molecule able to bind to a ligand that intervenes in a pathologic process. The term “substantially adds no further therapeutically beneficial property” further includes the meaning that the second polypeptide does not have erythropoietin biological activity or any of the activities of therapeutic peptides described in WO 00/69900. The second polypeptide should not be harmful. In particular, it should not have any harmful pharmacological effect and it should not be immunogenic, or at least have no more than a clinically acceptable low level of immunogenicity, particularly in humans.

Typically, therefore, the agent has substantially the same biological properties as albumin. For example, the agent may have substantially the same oncotic potential and/or transport functions (including detoxification) as albumin. The term “therapeutically inert” does not exclude a second polypeptide having the same, or at least some of the, therapeutic properties possessed by albumin. Thus an albumin-like polypeptide or a fragment thereof may be used as a second polypeptide. In a preferred embodiment, the second polypeptide may be a fragment of an albumin-like protein consisting essentially of one, two or more complete domains of albumin. Albumin has three complete domains, each containing two longer loops, separated by a shorter loop. Domains are usually numbered I, II and III, starting from the amino terminus. The structure of albumin, including HSA and BSA, is set out in Peters (1996, op. cit). An agent of the invention comprising an albumin-like polypeptide or a fragment thereof as a second polypeptide will be referred to hereinafter as an “albumin dimer”.

Thus it will be clear to the skilled person that an “albumin dimer” agent of the invention may comprise the albumin-like first polypeptide bound by its C-terminus to the N-terminus of the second polypeptide, or the second polypeptide bound by its C-terminus to the N-terminus of the albumin-like first polypeptide. Thus the albumin dimer may take the form of H₂N-[polypeptide 1]-X-[polypeptide 2]-COOH or H₂N-[polypeptide 2]-X-[polypeptide 1]-COOH, wherein polypeptide 1 is the albumin-like first polypeptide, polypeptide 2 is the second polypeptide that is also an albumin-like protein or fragment thereof. Thus the “dimer” may have two consecutive albumin-like sequences reading in the same direction. X may be any suitable form of bond. In one embodiment it may be a peptide bond. X may also be a linker molecule as described below. A preferred linker molecule is an oligopeptide, which may have from 1 to 20 amino acid residues. Where the linker is an oligopeptide it is usual for either, or preferably both, of polypeptide 1 and/or polypeptide 2 to be joined to the linker via a peptide bond.

An “albumin dimer” according to the invention may alternatively comprise the albumin-like first polypeptide bound by its N-terminus to the N-terminus of the second polypeptide or the albumin-like first polypeptide bound by its C-terminus to the C-terminus of the second polypeptide. These can be referred to as “head-to-head” and “tail-to-tail” dimers, respectively. Thus the “dimer” may have two consecutive albumin-like sequences reading in opposite directions. Optionally a linker may be included as described above. Such dimers can be prepared by conjugation of two albumin-like polypeptides, as described in more detail below.

The peptide bond may be dispensed with altogether provided that an appropriate linker moiety which retains the spacing between the Cα atoms of the amino acid residues is used; it is particularly preferred if the linker moiety has substantially the same charge distribution and substantially the same planarity as a peptide bond.

However, the present invention is not restricted to albumin dimers and any second polypeptide that meets the criteria set out above may be used. Typically, the second polypeptide comprises the full or partial sequence of a polypeptide naturally produced by the intended recipient of the agent. In a preferred embodiment, the second polypeptide comprises the full or partial sequence of albumin or a variant thereof. “Partial sequences” and “variants” with respect to second polypeptides will usually comprise at least 5%, 10%, 30%, 50%, 70%, 90%, 95% or 100% of the amino acids of the sequence fron which they were derived. Where a “partial sequence” and “variant” with respect to a second polypeptide comprises less than 100% of the amino acids of the sequence from which it is derived, the “partial sequence” and “variant” may be an N-terminal fragment, a C-terminal fragment or an internal fragment of the sequence from which it was derived. Typically “partial sequences” and “variants” with respect to second polypeptides will have at least at least 5%, 10%, 30%, 50%, 70%, 90%, 95% or 100% sequence identity to the sequence from which they were derived. Higher percentages with respect to length and, in particular, sequence identity are preferred because this tends to reduce the immunogenicity of the thus produced partial sequence or variant.

Second polypeptide bulking partners may be produced by any method known in the art. They may be natural proteins and purified from natural sources. They may be recombinantly expressed. They may be manufactured by synthetic processes. Techniques suitable to achieve all of these means of production are readily available to the skilled person. Polypeptides obtained by these techniques may be further modified, eg, chemically and/or enzymatically, by techniques known in the art.

In a preferred embodiment, the second polypeptide comprises the full or partial sequence of albumin or a variant thereof. Thus the second polypeptide may be a full-length albumin or one or two domains thereof, for example the albumin fragments. The second polypeptide may comprise the partial sequence of an albumin. In the latter case, it is preferred that the second polypeptide is bound to the first polypeptide which comprises the sequence of albumin in such a way as to avoid presenting any epitope not normally presented by full length albumin (e.g. an unnatural N- or C-terminus).

The albumin-like first polypeptide may be bound to the second polypeptide by any suitable bond known in the art. Typically the bond is covalent although it may be non-covalent (for example, see the description of the third aspect of the invention, below). Modified or synthetic amino acids may be included in the albumin-like polypeptide and/or the second polypeptide for this purpose. Linker molecules, eg, short alkyl group (C₁₋₃₀), oligopeptides, or the like may be provided to join the second polypeptide to the albumin-like first polypeptide. Suitable bonds include bonds commonly found in biologically derived macromolecules such as peptide bonds, disulphide bonds, phosphodiester linkages, ester linkages, ether linkages, thioester linkages, imine linkages, amine linkages and the like. Bi-functional cross-linkers can be provided to join a second polypeptide to the albumin-like polypeptide. For example, where the second polypeptide is a cysteine-containing protein then it is possible to use a bi-functional cross-linker that reacts with free sulphydryl groups, such as 1,4-bis-maleimidobutane (BMB), 1,4-bis-maleimidyl-2,3-dihydroxybutane (BMDB), bis-maleimidohexane (BMH), bis-maleimidoethane (BMOE), 1,8-bis-maleimidotriethyleneglycol (BM[POE]₃), 1,11-bis-maleimidotetra-ethyleneglycol (BM[POE]₄), 1,4-di[3′-(2′-pyridyldithio)-propionamido]-butane (DPDPB), dithio-bis-maleimidoethane (DTME) or 1,6-hexane-bis-vinylsulfone (HBVS) to join the albumin-like first polypeptide (e.g. via Cys34) to a proteinaceous bulking partner. Similarly, a tri-functional cross-linker such as Tris[2-maleimidoethyl]amine (TMEA) can be used to join the albumin-like first polypeptide to two proteinaceous bulking partners.

Typically the bond will be stable in the circulation system of a patient having NS. By “stable” is meant that the rate of loss of agent resulting from the dissociation of the bond is lower than the rate of loss of agent resulting from clearance of the intact agent from the blood stream via the kidney. Where a plurality of molecules of the agent of the first and/or second aspects of the invention are provided, typically at least 30%, 40%, 50%, 60%, 70%, 80%, 90% 95%, 98%, 99%, 99.5% or substantially 100% of those molecules comprise intact bonds between the albumin-like first polypeptide and the second polypeptide. Higher values are preferred.

In one embodiment, an agent of the first and/or second aspect of the invention comprises a bond between the albumin-like first polypeptide and the second polypeptide which bond is not a peptide bond. Any suitable non-peptide bond may be used. Typically the non-peptide bond will be a bond as discussed above.

However, in a preferred embodiment, an agent of the first and/or second aspect of the invention comprises an albumin-like first polypeptide bound to a second polypeptide via a peptide bond. Thus the agent may be a fusion protein. Typically a fusion protein comprises the sequence of an albumin-like first polypeptide fused via the N- or C-terminus directly or indirectly (ie, by a polypeptide linker) to the second polypeptide. Fusion proteins may be head-to-head or tail-to-tail fusion proteins, as described above in respect of albumin dimers, although the skilled person will appreciate that the principle is not limited to albumin dimers and that any other type of polypeptide sequence may be used as the second polypeptide so long as it meets the criteria provided above. The term “fusion protein” also includes with its meaning the inclusion of the second polypeptide at an internal position of the polypeptide which comprises the sequence of the albumin-like first polypeptide or the inclusion of the sequence of the albumin-like first polypeptide at an internal position of the second polypeptide. In a particularly preferred embodiment, the fusion protein comprises the albumin-like first polypeptide fused via its N- or C-terminal directly or indirectly to the N- or C-terminus of a second polypeptide, which bulking partner comprises the partial or full sequence of an albumin-like polypeptide.

The present invention is based on the appreciation that an increase in the size of an albumin-like molecule can increase the half-life of that molecule in a patient with a renal condition, such as NS.

In addition to providing albumin-like molecules with an increased size, as described above, it is also possible to modify the albumin molecules naturally produced by an individual by treating that patient directly, or treating an ex vivo albumin-containing sample taken from the patient, with an albumin ligand that binds to, and in doing so increases the size of, the albumin.

The albumin ligand will typically bind to albumin non-covalently. Any appropriate ligand may be used, provided that it specifically binds to albumin and that, having so bound, is therapeutically inert as defined above. By “specifically binds to albumin” is meant that the ligand must have a sufficiently high affinity for albumin such that, when used in a therapeutically effective amount to increase the half-life of albumin molecules in a patient and thereby ameliorate the effects of a renal disorder or condition associated therewith in the patient, then there is no significant undesirable side effect exhibited by the patient as a result of the binding of the albumin ligand to non-albumin molecules.

The albumin ligand may, for example, comprise the sequence of an antibody. Thus, for example, it may be an IgG molecule. In this context, the term antibody also includes fragments and portions thereof that retain sequences involved in binding.

The variable heavy (V_{H}) and variable light (V_{L}) domains of the antibody are involved in antigen recognition and binding, a fact first recognised by early protease digestion experiments. Further confirmation was found by “humanisation” of rodent antibodies.

Thus, a fragment may contain one or more of the variable heavy (V_{H}) or variable light (V_{L}) domains. For example, the term antibody fragment includes Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544).

Where the variable domain(s) used are not derived from the same species as the individual intended for treatment, the antibody may be modified by fusion of those variable domains(s) to constant domains derived from the species of individual intended for treatment. Thus, where a human is intended for treatment, the antibody may be “humanised” by fusion of the variable domain(s) to constant domains of human origin such that the resultant antibody retains the antigenic specificity of the parent antibody (for example, see Morrison et al (1984) Proc. Natl. Acad. Sci. USA 81, 6851-6855).

A general review of the techniques involved in the synthesis of antibody fragments which retain their specific binding sites is to be found in Winter & Milstein (1991) Nature 349, 293-299.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted recombinantly (e.g. from E. coli or yeast), thus allowing the facile production of large amounts of the said fragments.

The skilled person will be aware of alternatives to using antibodies. For example, the albumin ligand may comprise the amino acid sequence DICLPRWGCLW, a peptide sequence known to bind to albumin with high specificity (Dennis et al, 2002, J. Biol. Chem., 277: 35035), or a variant thereof which substantially retains the same albumin-binding properties.

In addition to comprising an albumin-binding region, the albumin ligand is typically large enough that, when bound to an albumin molecule, the albumin-albumin ligand complex has a greater half-life than naturally produced albumin, as defined above.

To aid in this, it may be suitable for the albumin ligand to comprise an albumin-binding region bound to a second polypeptide. The second polypeptide may be as defined above. For example, an albumin ligand may comprise an antibody as defined above fused to the full or partial sequence of albumin or a variant thereof.

Thus, the third aspect of the invention provides an albumin ligand having a size of from 14 to 200 kDa, more typically 14 to 150 kDa in size preferably at least 30 kDa in size. It may be preferable not to exceed 100 kDa. Around 60 to 70 kDa may be ideal. The molecular weight values are taken to be the ‘average’ molecular weight and are measured using (size exclusion chromatography) SEC-HPLC as described above.

An alternative to the albumin ligand having a size requirements as defined above is for the albumin ligand to further comprise a second binding site, i.e. for the albumin ligand to be bi-specific. Thus the albumin ligand may comprise two albumin binding regions, which may the same or different, so as to facilitate the formation of an albumin dimer. Alternatively, the albumin ligand may comprise, in addition to its albumin binding region, a second binding site capable of binding to another protein or other molecule, which protein or other molecule can act as a therapeutically inert bulking partner to provide the albumin-albumin ligand-bulking partner complex with a greater half-life.

The third aspect of the present invention also provides an albumin ligand as defined above for use in medicine, and for the use of an albumin ligand as defined above in the manufacture of a medicament for the treatment of a renal disorder or a condition associated therewith.

It is also possible to produce modified albumin, by recombinant or chemical methods, that includes a non-natural epitope. The non-natural epitope may, for example, be a C-terminal or N-terminal extension. It will then be possible to select an albumin ligand as defined above which has a binding specificity for the non-natural epitope, rather than a natural epitope of albumin. Binding of the albumin ligand to the modified albumin can then produce an agent according to the first aspect of the present invention.

Thus, the third aspect of the present invention also provides a system comprising, as a first component, a modified albumin comprising a non-natural epitope and, as a second component, an albumin ligand which binds specifically to the non-natural ligand of the modified albumin. Either or both of the first and second components may be provided in the form of composition formulated with a pharmaceutical acceptable carrier or diluent as defined above. The system can be used for medicine. For example, the system can be used for the treatment of a renal disorder or a condition associated therewith.

A fourth aspect of the invention provides a polynucleotide comprising a coding sequence that encodes an agent or an albumin ligand, in the form of a fusion protein, as defined above.

Furthermore, techniques are known in the prior art to modify endogenous genes in a cell by introduction of exogenous polynucleotide sequences into chromosomal DNA, e.g. by homologous recombination. For example, the reader is directed to the TKT system provided by Transkaryotic Therapies, Inc., as described in WO 01/68882. Using this system, double stranded DNA is introduced into a selected target DNA by homologous recombination by providing an agent which enhances homologous recombination (e.g. Rad52) and an agent which inhibits non-homologous end joining (e.g. a Ku inactivating agent) in close proximity to the target sequence.

Recombinant technology of this type can be used in the context of the present invention to modify albumin genes that are endogenous to a given cell. For example, a polynucleotide according to the fourth aspect of the present invention may be a double-stranded DNA suitable for use in the TKT system described in WO 01/68882 comprising a sequence suitable for introducing a non-natural epitope into an albumin gene expression product, thereby to produce a cell that expresses modified albumin as described in the third aspect of the invention.

A polynucleotide according to the fourth aspect of the invention may be a double stranded DNA suitable for use in the TKT system as described in WO 01/68882 comprising a sequence suitable for introducing a second polypeptide bulking partner as defined above into an albumin gene expression product, thereby to produce a cell that expresses an agent of the invention as a fusion protein.

Whilst the following passages pertain to polynucleotides of the fourth aspect of the invention, the skilled person will also appreciate that polynucleotides encoding a polypeptide comprising the sequence of albumin in the absence of a fused second polypeptide may be produced and expressed using equivalent techniques (a thus produced albumin protein being useful in the production of non-fusion protein agents of the invention). The polynucleotide is typically a DNA or RNA. Polynucleotides of the invention may be produced by any method known in the art, such as by recombinant methods or synthetic methods (Sambrook and Russell (2001) Molecular Cloning, A Laboratory Manual, (3rd Ed) Cold Spring Harbor Laboratory Press, Cold Spring Harbor, N.Y.). For example, polynucleotides according to the fourth aspect of the invention may be produced by synthetic methods well known in the art, usually utilising a solid-support such as controlled-pore glass or polystyrene (for a review see Sambrook and Russell (2001) op. cit. and the references therein). Automated synthetic machines are available for this purpose and custom-synthesised polynucleotides of defined sequence can be obtained commercially from a number of companies. Synthetic polynucleotides may be synthesised by producing short oligonucleotides and combining them to produce full length polynucleotides according to the fourth aspect of the invention using techniques known in the art, such as by ligation or polymerase chain reaction (PCR). Additionally and/or alternatively, polynucleotides according to the fourth aspect of the invention may comprise DNA or RNA derivable directly or indirectly from natural sources by techniques well known in the art. Thus a polynucleotide according to the fourth aspect of the invention may comprise a combination of naturally derived and synthetic polynucleotide sequences. Moreover, the sequence of naturally derived polynucleotides may be modified, for example, to optimise codon usage in accordance with the intended means of expression, such as to suit the chosen host cell or in vitro expression system. Additional sequences may be included in a polynucleotide according to the fourth aspect of the invention, such as signal or leader sequences, which may, for example, cause secretion of the expressed protein product of the polynucleotide according to the fourth aspect of the invention. Secretion may be particularly desirable for the purposes of harvesting a protein from an expression system, such as a culture of recombinant cells expressing a polynucleotide according to a fourth aspect of the invention, since harvesting a secreted polypeptide from the culture medium may be more efficient than harvesting non-secreted polypeptide from within a recombinant cell.

The sequence of the thus produced polynucleotides can be confirmed by any suitable method, such as by sequencing. The skilled person will appreciate that it is possible to introduce synthetic or modified nucleotides into a polynucleotide according to the fourth aspect of the invention, for example to increase the stability (reduce the in vitro or in vivo degradation) or to modify the secondary structure (which may, for example, allow modulation of the in vitro or in vivo expression of the polynucleotide) of the thus produced polynucleotide.

Usually a polynucleotide according to the fourth aspect of the invention further comprises a regulatory region operatively linked to the coding sequence. The term “regulatory region” refers to a sequence that modulates (ie, promotes or reduces) the expression (ie, the transcription and/or translation) of a coding region to which it is operably linked, in this case the coding region encoding a fusion protein according to the first and/or second aspect of the invention or the coding region encoding an albumin ligand according to the third aspect of the invention. Regulatory regions typically include promoters, terminators, ribosome binding sites and the like. The skilled person will appreciate that the choice of regulatory region will depend upon the intended expression system. For example, promoters may be constitutive or inducible and may be cell- or tissue-type specific or non-specific. Where the polynucleotide according to the fourth aspect of the invention is a DNA, it may be beneficial to incorporate more than one DNA sequence encoding a translational stop codon, such as UAA, UAG or UGA, in order to minimise translational read-through and thus avoid the production of elongated, non-natural fusion proteins. A DNA sequence encoding the translation stop codon UAA is preferred. The skilled person will understand that equivalent sequences can be introduced into RNA polynucleotides.

The term “operably linked” includes within its meaning that a regulatory region is positioned within a polynucleotide according to the fourth aspect of the invention such that it forms a relationship with a coding sequence, such as a sequence encoding a fusion protein, that permits the regulatory region to function in its intended manner. Thus a regulatory region “operably linked” to a fusion protein coding sequence is positioned in such a way that the regulatory region is able to influence transcription and/or translation of the coding sequence in the intended manner, under conditions compatible with the regulatory region.

A polynucleotide according to the fourth aspect of the invention may be introduced into a vector. The skilled person will appreciate that different types of vectors are suitable for use in different applications. The vector may be, for example, a plasmid suitable for expression in bacterial or yeast cells. The vector may be a viral vector. Typical vectors include cloning vectors and expression vectors. When vectors are introduced into a host cell, the vector may remain as an extrachromosomal polynucleotide or become integrated into the chromosome of the host cell.

Thus, the vector may be introduced into a host through standard techniques, followed by selection for transformed host cells. Host cells so transformed are then cultured for a sufficient time and under appropriate conditions known to those skilled in the art, and in view of the teachings disclosed herein, to permit the expression of the coding sequence of the polynucleotide according to the fourth aspect of the invention, which can then be recovered.

Many expression systems are known, including bacteria (for example E. coli and Bacillus subtilis), yeasts (for example Saccharomyces cerevisiae, Pichia pastoris and Kluyveromyces lactis), filamentous fungi (for example Aspergillus), plant cells, whole plants, animal cells and insect cells.

In one embodiment the preferred host cells are the yeasts Saccharomyces cerevisiae, Kluyveromyces lactis and Pichia pastoris. It is particularly advantageous to use a yeast deficient in one or more protein mannosyl transferases involved in O-glycosylation of proteins, for instance by disruption of the gene coding sequence.

The albumin protein sequence does not contain any sites fox N-linked glycosylation and has not been reported to be modified, in nature, by O-linked glycosylation. However, it has been found that rHA produced in a number of yeast species can be modified by O-linked glycosylation, generally involving mannose. The mannosylated albumin is able to bind to the lectin Concanavalin A. The amount of mannosylated albumin produced by the yeast can be reduced by using a yeast strain deficient in one or more of the PMT genes (WO 94/04687). The most convenient way of achieving this is to create a yeast which has a defect in its genome such that a reduced level of one of the Pmt proteins is produced. For example, there may be a deletion, insertion or transposition in the coding sequence or the regulatory regions (or in another gene regulating the expression of one of the PMT genes) such that little or no Pmt protein is produced. Alternatively, the yeast could be transformed to produce an anti-Pmt agent, such as an anti-Pmt antibody.

If a yeast other than S. cerevisiae is used, disruption of one or more of the genes equivalent to the PMT genes of S. cerevisiae is also beneficial, eg in Pichia pastoris or Kluyveromyces lactis. The sequence of PMT1 (or any other PMT gene) isolated from S. cerevisiae may be used for the identification or disruption of genes encoding similar enzymatic activities in other fungal species. The cloning of the PMT1 homologue of Kluyveromyces lactis is described in WO 94/04687.

The yeast will advantageously have a deletion of the HSP15O and/or YAP3 genes as taught respectively in WO 95/33833 and WO 95/238 57.

In a preferred embodiment the yeast is transformed with an expression plasmid based on the Saccharomyces cerevisiae 2 μm plasmid. At the time of transforming the yeast, the plasmid contains bacterial replication and selection sequences, which are excised, following transformation, by an internal recombination event in accordance with the teaching of EP 286 424. The plasmid also contains an expression cassette comprising: a yeast promoter (eg the Saccharomyces cerevisiae PRB1 promoter), as taught in EP 431 880; a sequence encoding a secretion leader which, for example, comprises most of the natural HSA secretion leader, plus a small portion of the S. cerevisiae α-mating factor secretion leader as taught in WO 90/01063; the HSA coding sequence, obtainable by known methods for isolating cDNA corresponding to human genes, and also disclosed in, for example, EP 73 646 and EP 286 424; and a transcription terminator, preferably the terminator from Saccharomyces ADH1, as taught in EP 60 057. Preferably, the vector incorporates at least two translation stop codons.

The choice of various elements of the plasmid described above is not thought to be directly relevant to the purity of the albumin product obtained, although the elements may contribute to an improved yield of product.

In another embodiment the host cell is animal cell. Typically the animal cell is a mammalian cell, preferably a mammalian liver cell. In this embodiment the preferred cell type is a human cell type.

Thus a fifth aspect of the invention provides a host cell as described above comprising a polynucleotide according to the fourth aspect of the invention or a vector containing a polynucleotide according to the fourth aspect of the invention.

The host cell may be directly therapeutic (ie, capable of providing therapeutic effects when contacted with the recipient). Fox example, the host cell may be suitable for implantation into a recipient in need of an agent fusion protein of the invention. The recipient may be a human. The recipient may have nephrotic syndrome. Additionally and/or alternatively, the host cell may provide a means of expressing and obtaining polypeptides useful in the present invention, eg, the host cell may be used in the recombinant production of polypeptides such as fusion proteins or components for the manufacture of non-fusion protein agents according to the first or second aspects of the invention or an albumin ligand according to the third aspect of the invention. Polypeptide obtained in this manner may optionally be further modified (eg, by linking to a second polypeptide) and/or optionally further purified as discussed herein.

Thus, a sixth aspect of the invention provides a method for producing an agent or an albumin ligand as defined by the first, second or third aspects of the invention comprising the steps of obtaining a cell according to the fifth aspect of the invention and isolating therefrom a fusion protein comprising a polypeptide which comprises the sequence of an albumin-like first polypeptide fused to the sequence of the second polypeptide.

Purification techniques for rHA have been disclosed in: WO 92/04367, removal of matrix-derived dye; EP 464 590, removal of yeast-derived colorants; and EP 319 067, alkaline precipitation and subsequent application of the rHA to a lipophilic phase having specific affinity for albumin. The skilled person will appreciate that these methods can be applied to the isolation of a fusion protein or albumin ligand according to the first, second or third aspects of the invention. In a preferred embodiment, the sixth aspect of the invention involves isolating a fusion protein by a method described in WO 00/44772 or WO 96/37515, both of which are incorporated herein by reference.

The terms “albumin solution” and “albumin”, as used below with reference to purification, are intended to encompass an albumin/fusion-protein solution and albumin/fusion-protein, respectively, such as is obtainable from a cell according to a fifth aspect of the invention.

A purification process used in a method according to the sixth aspect of the invention may, for example, comprise the steps of applying a relatively impure albumin solution to a chromatographic material for which the albumin has no specific affinity such that albumin binds to the material, and eluting the bound albumin from the material by applying a solution of a compound having a specific affinity for albumin. Preferably, the chromatographic material is a cation exchanger, such as SP-Sepharose FF, SP-Spherosil etc. The compound with specific affinity for albumin may be octanoate (eg sodium octanoate), other long chain (C₆ to C₂₂) fatty acids, salicylate, octylsuccinate, N-acetyltryptophan or a mixture of two or more of these.

The final product may be formulated to give it added stability. Preferably, the highly pure albumin, such as an albumin fusion protein of the invention or an albumin protein for use in the manufacture of a non-fusion protein agent of the invention may contains at least 100 g, more preferably 1 kg or 10 kg of albumin-fusion protein or albumin, which may be split between a plurality of vials.

However, where the purified albumin is not the final product, but merely a precursor to the final product, further modification is required to obtain an agent or albumin ligand according to the first, second or third aspect of the invention. Hence, a seventh aspect of the invention provides a method for producing an agent having a greater half-life than naturally produced albumin in a patient with NS, the agent comprising a first albumin-like polypeptide bound to a second polypeptide, wherein the second polypeptide is therapeutically inert, comprising the steps of:

(a) providing, as a first component, an albumin-like first polypeptide;

(b) providing, as a second component, the second polypeptide; and

(c) contacting the first component with the second component under conditions suitable to allow the formation of the agent.

By “conditions suitable to allow the formation of the agent” we include any conditions under which the second polypeptide becomes bound to the first polypeptide that comprises the albumin-like sequence without causing irreversible changes to either the second polypeptide or the first albumin-like polypeptide which irreversible changes render the changed molecule toxic to, or ineffective in, the recipient. The skilled person will appreciate that conditions can be adapted to suit the formation of the desired type of bond, taking into account the identities of the first and second components. The thus formed agent may be further purified, for example, using the techniques discussed above.

The skilled person will appreciate that it is possible to use an equivalent method to produce an albumin ligand as defined above by contacting an albumin-binding molecule with a second polypeptide under conditions suitable to allow the formation of the albumin ligand.

Methods to conjugate a polypeptide to another polypeptide are well known in the art. For example, Proteins-LabFax, (N. C. Price, 1996, Academic Press) describes a protocol of crosslink polypeptides through free SH groups using homobifunctional cross-linking reagents like 4,4′-dimaleimidostylbene, or through free —SH groups and —NH2 groups using heterobifunctional cross-linking reagents like N-succinimidyl 6-maleimidocaproate, or through —NH₂ groups using homobifunctional cross-linking reagents like disuccinimidylsuberate. U.S. Pat. No. 4,136,093 describes the cross-linking of polypeptides through the —NH₂ groups using glutaraldehyde. Other bi-functional and tri-functional crosslinkers are discussed above.

An eighth aspect of the invention provides a method for producing a composition according to the second aspect of the invention, or a composition comprising an albumin ligand as defined by the third aspect of the invention, comprising producing an agent or albumin ligand by a method according to sixth or seventh aspect of the invention and formulating the agent or albumin ligand with a pharmaceutically acceptable carrier or diluent.

The following example illustrates pharmaceutical formulations according to the invention in which the active ingredient is an agent or albumin ligand of the first, second or third aspects of the invention.

Injectable Formulation

| | | |
|---|---|---|
| Active ingredient | 10 | g |
| Sterile, pyrogen free phosphate buffer (pH 7.0) | to 50 | ml |

The active ingredient is dissolved in most of the phosphate buffer (20-40° C.), then made up to volume and filtered through a sterile 0.2 μm filter into a sterile 50 ml glass vial (type I or II) and sealed with sterile closures and overseals. The reader will appreciate that the amount of active ingredient can be increased or decreased to suit the condition being treated. For example, the amount of active ingredient used can be greater than 10 g, such as 20, 30, 40, 50, 60, 70, 80, 90, 100, 200, 300, 400, 500 or more grams. The volume of the formulation can be adapted to ensure the active ingredient is provided at a physiologically acceptable concentration.

Formulations suitable for parenteral administration include aqueous sterile injection solutions which may contain anti-oxidants, buffers, albumin stabilisers and solutes which render the formulation isotonic with the blood of the intended recipient. The formulations may be presented in unit-dose or multi-dose containers, for example sealed ampoules and vials, and may be stored in a freeze-dried (lyophilised) condition requiring only the addition of the sterile-liquid carrier, for example water for injections, immediately prior to use. Extemporaneous injection solutions may be prepared from sterile powders of the kind previously described.

A suitable injectable formulation may be analysed to determine the concentration of the agent of the invention, of sodium and of octanoate. These quantities are taken into account and any necessary further amounts of stock sodium chloride and sodium octanoate excipient solutions and appropriate grade water added to achieve the formulation specification. The final agent concentration may be 150-250 g.L⁻¹ or 235-265 g.L⁻¹, with a sodium concentration of 130-160 mM. Any other feasible agent concentration may be made, however, with, for example, a minimum concentration of at least 4% (w/v), preferably 4-25 % (w/v). The formulation may also include appropriate conventional pharmaceutically acceptable excipients, such as polysorbate 80 or those specified in the US Pharmacopoeia for human albumin, and diluting water.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other components conventional in the art having regard to the type of formulation in question.

Thus an agent, composition or albumin ligand of the first, second or third aspects of the invention or obtainable by a method of any one of the sixth, seventh or eighth aspects of the invention or a polynucleotide or vector according to the fourth aspect of the invention or a cell according to the fifth aspect of the invention may be administered to bring about a therapeutically beneficial effect to a recipient. By “therapeutically beneficial” is included the meaning that the effects of a renal disorder, such as nephrotic syndrome (NS) or uremia, or a condition associated therewith, are ameliorated. Conditions associated with renal disorders include conditions associated with the symptoms or renal disorders. Such symptoms include hypoalbuminemia, proteinuria, albuminuria, oedema, low plasma volume and hyperlipidemia. An example, therefore, of a condition associated with a renal disorder, is atherosclerotic cardiovascular disease, which is known to be linked to hyperlipidemia.

Typically agents, albumin ligands, compositions, polynucleotides, vectors and cells of the invention or a formulation thereof will be used in methods as described herein wherein the recipient has a renal disorder. In one preferred embodiment the recipient may have nephrotic syndrome. In another preferred embodiment the recipient may have uremia. Additionally and/or alternatively, the recipient may have a condition associated with a renal disorder, such as oedema or atherosclerotic cardiovascular disease. The use of agents, albumin ligands, compositions, polynucleotides, vectors and cells of the invention or a formulation thereof in methods as described above can thus provide treatment for hypoproteinemia, hypoalbuminemia, proteinuria, hyperlipidemia, oedema, and provide a means to maintain plasma colloidal pressure and improve oxygen distribution to tissues.

Nephrotic syndrome is characterised by severe leakiness of the glomerular filtration system. Agents or albumin ligands of known molecular weights according to the invention may be administered to an individual and the molecular weight threshold for leakiness of the individual's glomerular filtration system be determined. Agents or albumin ligands of the invention may be labelled using techniques well know in the art. Following administration of the labelled agents or albumin ligands of the invention to an individual, the step of attempting to detect excretion of the labelled agents or albumin ligands may then be performed, for example, by analysis of the urine of the individual. Agents or albumin ligands administered may have same molecular weight as each other. Alternatively, different sized agents or albumin ligands, optionally having different labels corresponding to each different size may be administered simultaneously. Detection of agents or albumin ligands in, for example, the urine of the individual can indicate the molecular weight threshold for excretion of the individual's glomerular filtration system. Of course, if the size of the albumin ligand is, itself, below the molecular weight threshold of the patient's glomerular filtration then it will be necessary to determine whether the excreted albumin ligand is excreted as a free ligand or when bound to albumin, since then only excretion when bound to albumin would be indicative of a ‘leaky’ glomerular filtration system. Thus agents and albumin ligands of the invention may be used in a method of diagnosing conditions associated with abnormal glomerular filtration system function, such as NS.

Accordingly a ninth aspect of the invention provides an agent, composition or albumin ligand of the first, second or third aspects of the invention or obtainable by a method of any one of the sixth, seventh or eighth aspects of the invention or a polynucleotide or vector according to the fourth aspect of the invention or a cell according to the fifth aspect of the invention for use in a method for treatment of the human or animal body by surgery, therapy or diagnosis.

As discussed above, agents, albumin ligands, compositions thereof, polynucleotides, vectors and cells can be formulated in a manner suitable for administration to a recipient. Thus, in a tenth aspect of the invention, there is provided the use of an agent, composition or albumin ligand according to the first, second or third aspect of the invention or obtainable by a method of any one of the sixth, seventh or eighth aspects of the invention or a polynucleotide or vector according to the fourth aspect of the invention or a cell according to the fifth aspect of the invention in the manufacture of a medicament for treating a renal disorder or a condition associated therewith.

The thus formulated medicament may then be administered to the recipient. Accordingly, an eleventh aspect of the invention provides a method for treating a patient with a renal disorder or a condition associated therewith comprising administering to the patient an agent, composition or albumin ligand as according to the first, second or third aspects of the invention or obtainable by a method of any one of the sixth, seventh or eighth aspects of the invention or a polynucleotide or vector according to the fourth aspect of the invention or a cell according to the fifth aspect of the invention in a pharmaceutically effective amount and concentration.

The aforementioned agents, albumin ligands, compositions, polynucleotides, vectors and cells of the invention or a formulation thereof may be administered by any conventional method including oral and parenteral (eg subcutaneous or intramuscular) injection. The treatment may consist of a single dose or a plurality of doses over a period of time. Although it is possible for an agent, albumin ligand, polynucleotide, vector or cell of the invention to be administered alone, it is preferable to present it as a pharmaceutical formulation, together with one or more acceptable carriers.

As used herein, the term “pharmaceutically effective amount” includes within its meaning the optimum amount of agent, albumin ligand or compositions of the invention to be administered to the individual. This can be determined by a person skilled in the art taking into account the species, size, age and health of the patient or subject. The optimum amount will be the amount that is best able to ameliorate the effects of a renal disorder or a condition associated therewith without causing unacceptable levels unwanted side effects, such as cytotoxic effects or immune responses. The skilled person will be able to determine whether a dose has an unacceptable side effect. The amount of agent or composition to be administered in a single dose is typically from 1 g to 200 g, more typically from 5 g to 150 g, preferably from 10 g to 100 g. As a general proposition, the total pharmaceutically effective amount of agent administered parenterally, per dose will be in the range of 0.5 g/kg to 5 g/kg of a patient body-weight. Generally, one dose per day, or every 3 or 7 days, is given, effective to maintain the normal amount of albumin in the patient.

The invention will now be described in more detail by reference to the following Figures and Examples wherein:

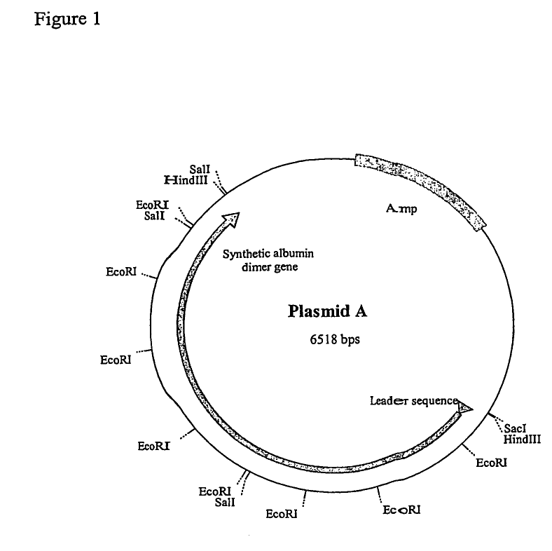

FIG. 1 shows a plasmid map of plasmid A.

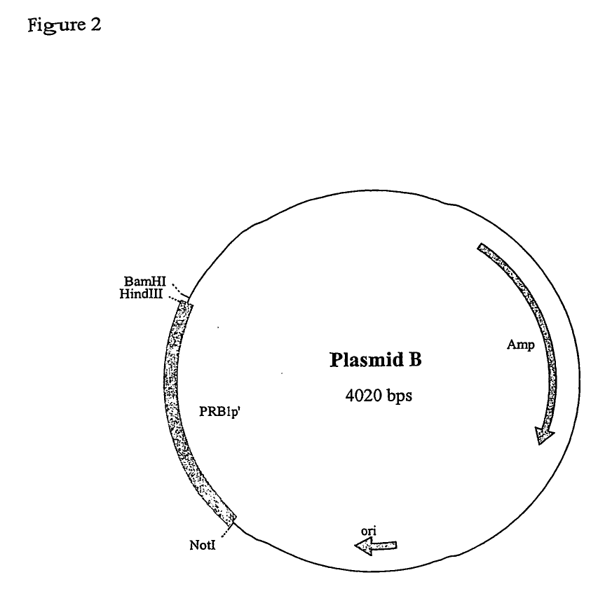

FIG. 2 shows a plasmid map of plasmid B.

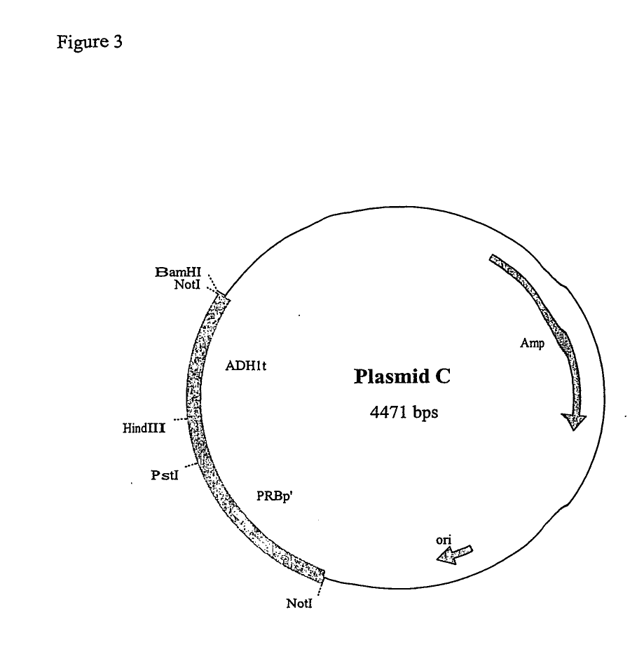

FIG. 3 shows a plasmid map of plasmid C.

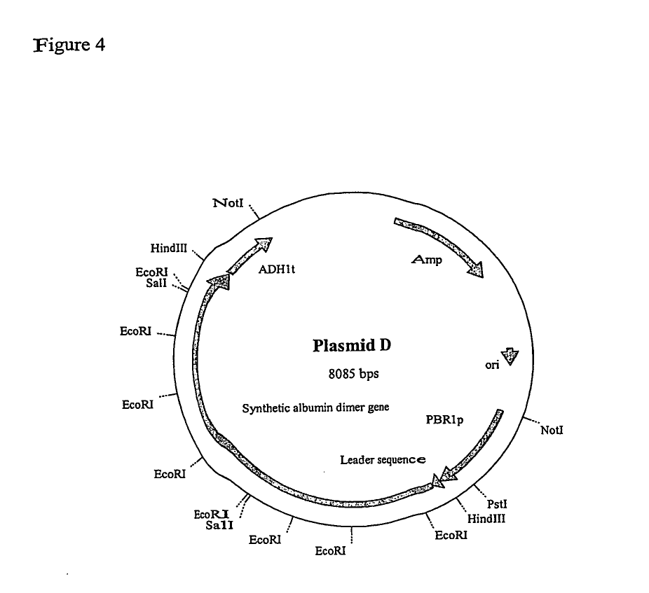

FIG. 4 shows a plasmid map of plasmid D.

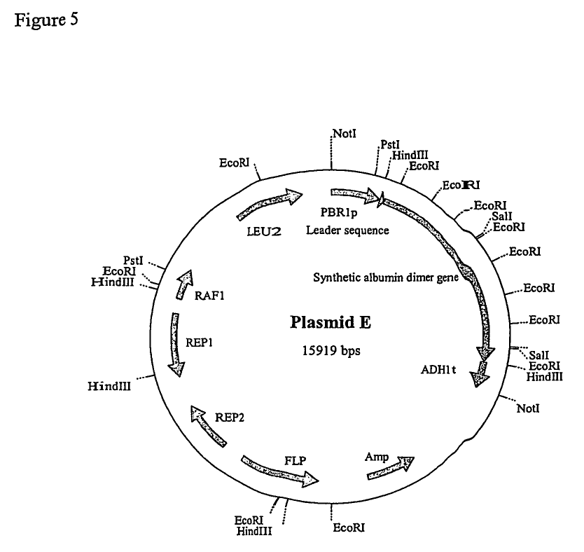

FIG. 5 shows a plasmid map of plasmid E.

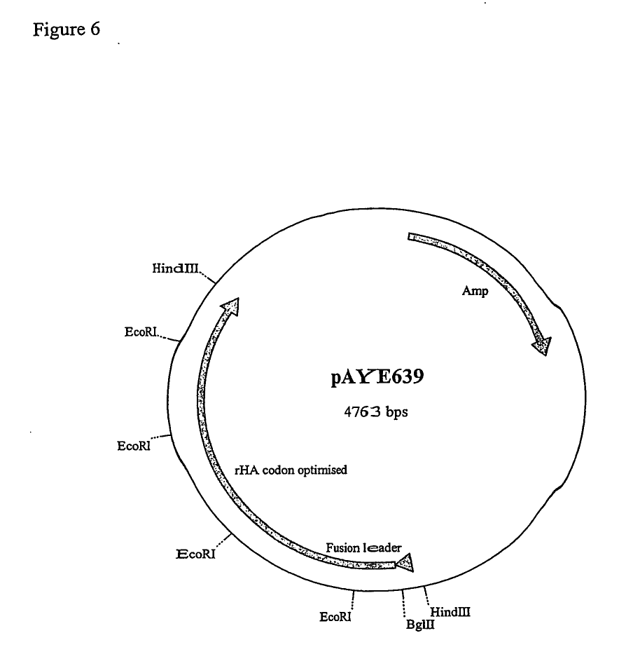

FIG. 6 shows a plasmid map of plasmid pAYE639.

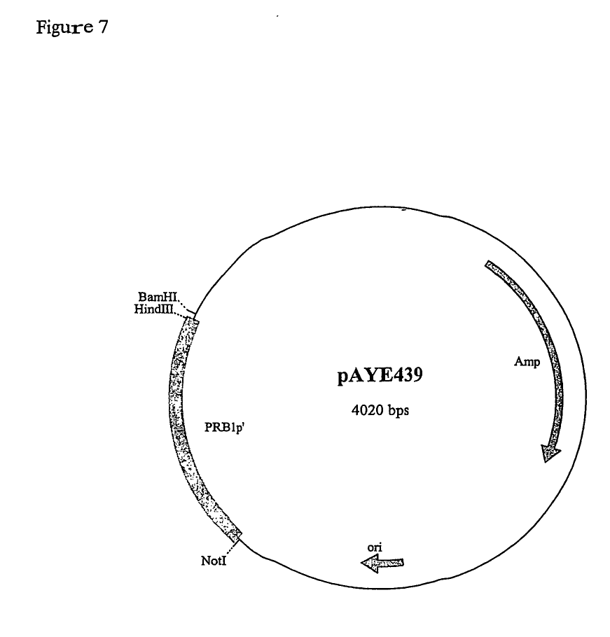

FIG. 7 shows a plasmid map of plasmid pAYE439.

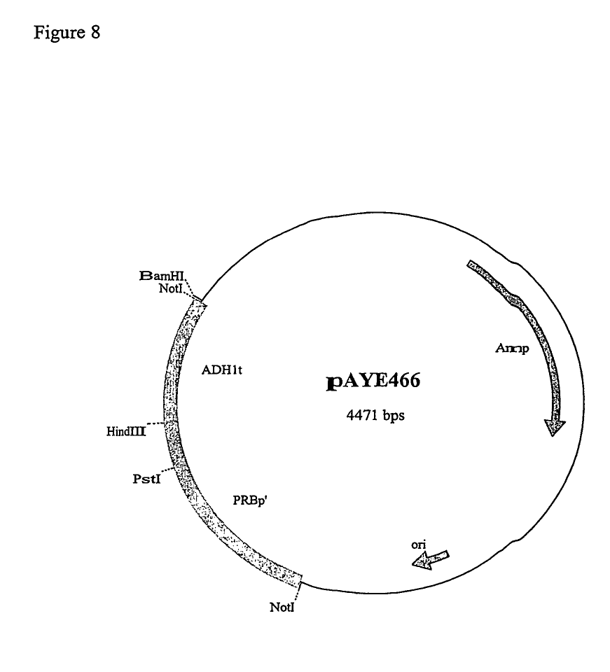

FIG. 8 shows a plasmid map of plasmid pAYE466.

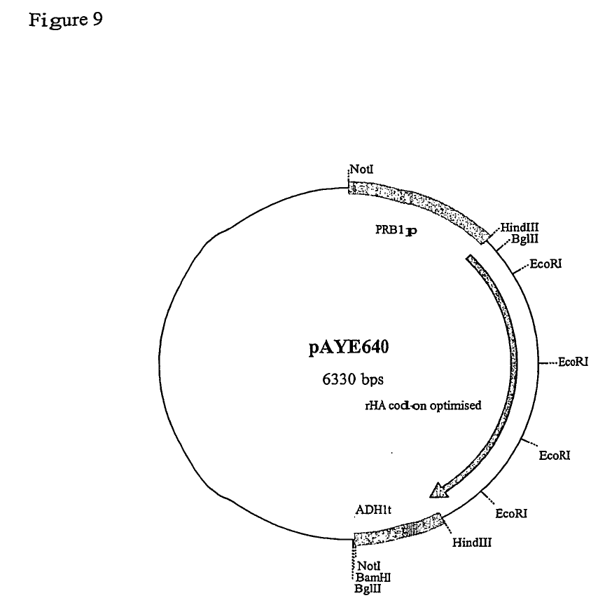

FIG. 9 shows a plasmid map of plasmid pAYE640.

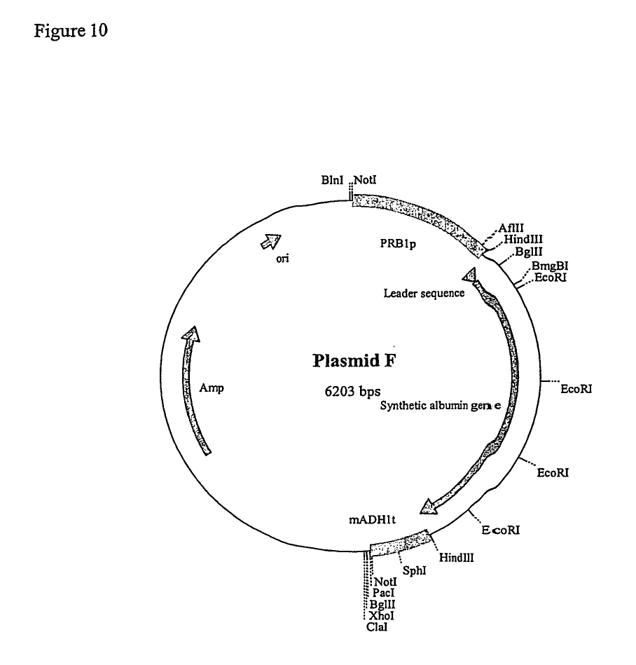

FIG. 10 shows a plasmid map of plasmid F.

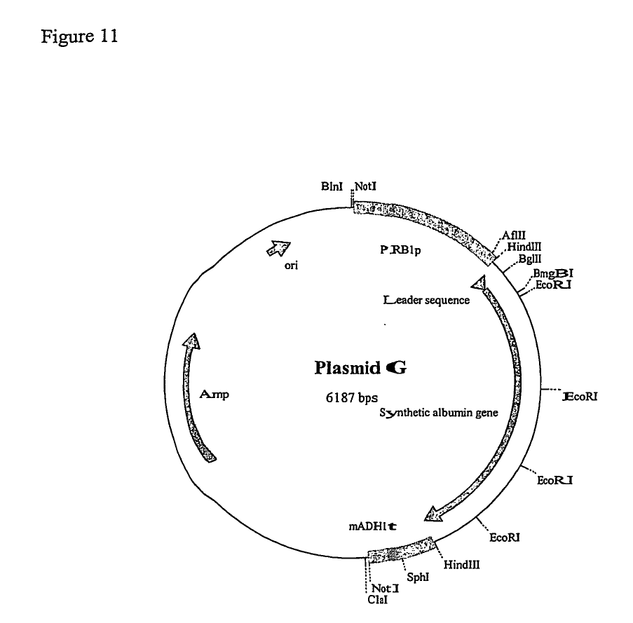

FIG. 11 shows a plasmid map of plasmid G.

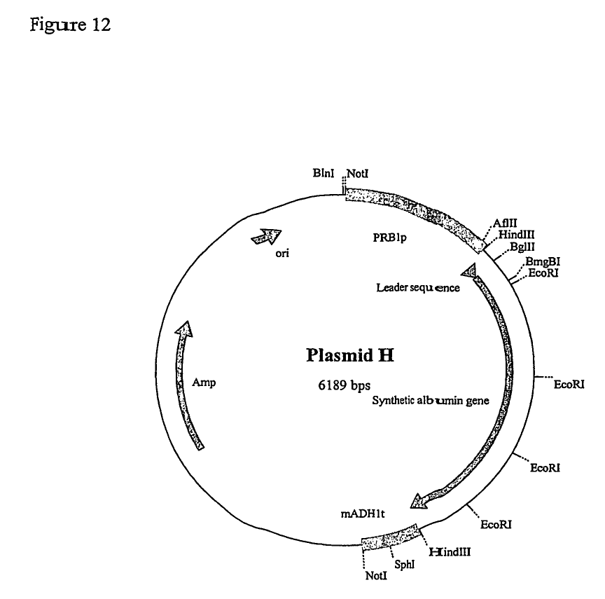

FIG. 12 shows a plasmid map of plasmid H.

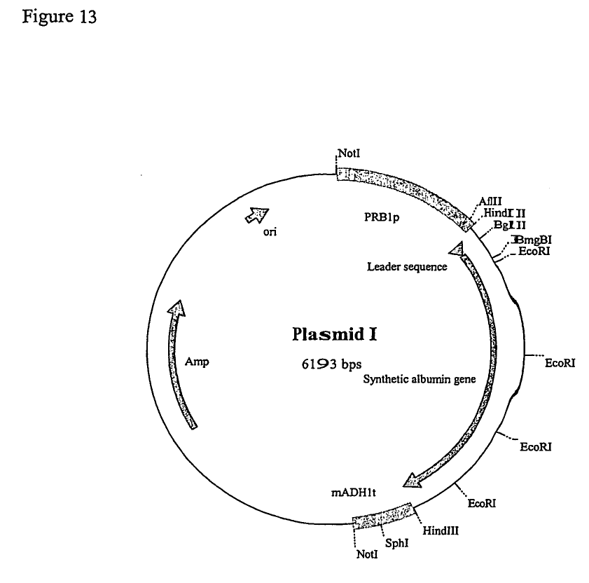

FIG. 13 shows a plasmid map of plasmid I.

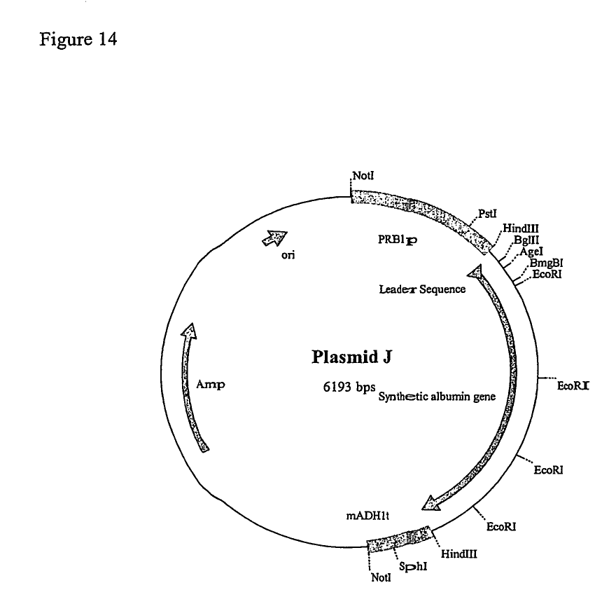

FIG. 14 shows a plasmid map of plasmid J.

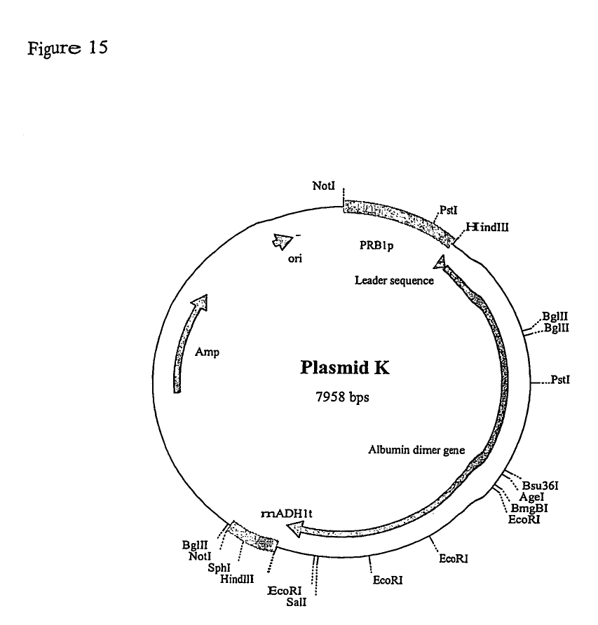

FIG. 15 shows a plasmid map of plasmid K.

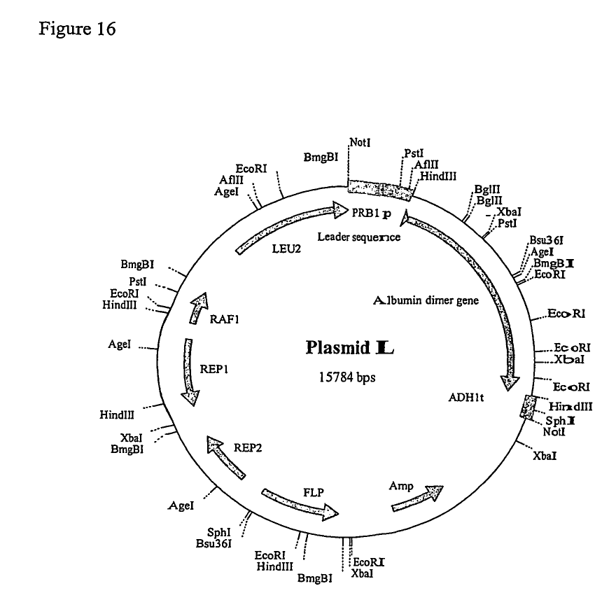

FIG. 16 shows a plasmid map of plasmid L.

### EXAMPLE 1

SEQ ID NO:5 shows a DNA sequence encoding an agent according to the present invention, comprising: a non-coding region that includes a 5′ UTR from the Saccharomyces cerevisiae PRB1 promoter (SEQ ID NO:1); a polynucleotide region encoding an HSA/MF_{α}-1 fusion leader sequence as defined in WO 90/01063 (SEQ ID NO:2); a first codon optimised coding region for mature human albumin fused to a second codon optimised coding region for mature human albumin (both SEQ ID NO:3) and translation termination sites (SEQ ID NO:4).

Such DNA sequences are obtainable from Genosys, Inc (Cambridge, UK) in the form of overlapping single-stranded oligonucleotides.

SEQ ID NO:5 is synthesised as a SacI-HindIII DNA fragment and cloned into the SacI-HindIII sites of plasmid pBSSK-(Stratagene Europe, P.O. Box 12085, Amsterdam, The Netherlands), as plasmid A (FIG. 1).

The Saccharomyces cerevisiae PRB1 promoter is isolated from yeast genomic DNA by PCR using two single stranded oligonucleotides PRBJM1 (SEQ ID NO:6) and PRBJM3 (SEQ ID NO:7).

The PCR conditions are 40 cycles of 94° C. for 30 seconds, 50° C. for 40 seconds, 72° C. for 120 seconds, followed by 72° C. for 600 seconds, followed by a 4° C. hold. The 0.81 kb DNA fragment is digested with both NotI and HindIII and ligated into pBST+, described in WO 97/24445, similarly digested with NotI and HindIII, to create plasmid B (FIG. 2). Plasmid B was digested with HindIII and BamHI and ligated with the 0.48 kb HindIII/BamHI ADH1 terminator DNA fragment from pAYE440 previously disclosed in WO O0/44772, so as to create plasmid C (FIG. 3).

The 3.6 kb HindIII DNA fragment of SEQ ID NO:5 was cloned into the unique HindIII site of plasmid C to create plasmid D, which can be shown to contain the appropriately sized HindIII DNA fragment of SEQ ID NO: 5 between the PRB1 promoter and the ADH1 terminator in the correct orientation for expression from the PRB1 promoter (FIG. 4).

Plasmid D is digested to completion with NotI and the NotI PRB1 promoter/HindIII DNA fragment of SEQ ID NO:5 gene/ADH1 terminator expression cassette is purified. The NotI, expression cassette from plasmid D is ligated into NotI linearised pSAC35 (Sleep et al. (1991), Bio/technology 9: 183-187), which is previously treated with calf intestinal phosphatase (CIP) to create plasmid E (FIG. 5). Plasmid E can be shown to contain the NotI expression cassette inserted into the NotI site of pSAC35 and orientated so that the expression of the fusion gene is away from the LEU2 auxotrophic marker and toward the 2 μm origin of replication.

A yeast strain is transformed to leucine prototrophy with plasmid E by the method described by Hinnen et al, (1978) P.N.A.S. 75, 1929. The transformants are patched out onto Buffered Minimal Medium (BMM, described by Kerry-Williams, S. M. et al. (1998) Yeast 14, 161-169) containing 2% (w/v) glucose (BMMD) and incubated at 30° C. until grown sufficiently for further analysis. The productivity of albumin fusion protein by the transformants is analysed from 10 mL YEP (1% (w/v) yeast extract; 2% (w/v) bacto peptone) containing 2% (w/v) glucose (YEPD) and BMMD shake flask culture (30° C., 2O0 rpm, 72 hr) by rocket immunoelectrophoresis of cell free culture supernatant.

A stock master cell culture of transformants demonstrating acceptable levels of albumin fusion protein production is used to prepare running stocks (working cell bank) of process yeast suitable for the preparation of shake flask cultures by freezing aliquots of the culture in the presence of 20% (w/v) trehalose.

The fermentation is essentially the same as is described in WO 00/44772 which is incorporated herein by reference.

The centrate from the fermentation is prepared, or conditioned, for chromatography on a cation exchange matrix essentially as described in WO 00/44772. The purification process comprises the following steps: cation exchange chromatography; anion exchange chromatography; affinity chromatography; ultrafiltration and diafiltration; a second affinity chromatography step; ultrafiltration and diafiltration; a second cation exchange chromatography step; and a second anion exchange chromatography step essentially as described in WO 00/44772. Preferably, these purification processes are followed by final ultrafiltration/diafiltration followed by a formulation step including the addition of water for injection in order to achieve the desired protein concentration, e.g. 200 g/L, the addition of known albumin stabilisers like sodium octanoate and/or polysorbate 80, the addition of a solute (e.g. NaCl) to achieve isotonicity with the blood of the recipient, and adjusting the pH to physiological compatibility, e.g. pH 7.0, and/or placing of the solution into a final container.

### EXAMPLE 2

SEQ ID No 8 shows a DNA sequence that comprises: a non-coding legion that includes a 5′ UTR from the Saccharomyces cerevisiae PRB1 promoter; a polynucleotide region encoding a HSA/MFα-1 fusion leader sequence; a codon optimised coding region for mature human albumin and translation termination sites.

The DNA sequence was synthesised by Genosys, Inc (Cambridge, UK) from overlapping single-stranded oligonucleotides as a 1.865 kb SacI-HindIII DNA fragment cloned into the SacI-HindIII sites of plasmid pBSSK-(Stratagene Europe, P.O. Box 12085, Amsterdam, The Netherlands), as plasmid pAYE639 (FIG. 6).

The Saccharomyces cerevisiae PRB1 promoter was isolated from yeast genomic DNA by PCR using two single stranded oligonucleotides PRBJM1 (SEQ ID NO:9) and PRBJM3 (SEQ ID NO:10).

The PCR conditions 40 cycles of 94° C. for 30 seconds, 50° C. for 40 seconds, 72° C. for 120 seconds, followed by 72 ° C. for 600 seconds, followed by a 4° C. hold. The 0.81 kb DNA fragment was digested with both NotI and HindIII and ligated into pBST+, described in WO 97/24445, similarly digested with NotI and HindIII, to create plasmid pAYE439 (FIG. 7). Plasmid pAYE439 was digested with HindIII and BamHI and ligated with the 0.48 kb HindIII/BamHI ADH1 terminator DNA fragment from pAYE440 previously disclosed in WO 00/44772, so as to create plasmid pAYE466 (FIG. 8).

A 1.865 kb HindIII DNA fragment of SEQ ID No 7 was cloned into the unique HindIII site of plasmid pAYE466 to create plasmid pAYE640, which was shown to contain the 1.865 kb HindIII DNA fragment of SEQ ID No 26 between the PRB1 promoter and the ADH1 terminator in the correct orientation for expression from the PRB1 promoter (FIG. 9).

Plasmid pAYE640 contains the yeast PRB1 promoter and the yeast ADH1 terminator providing appropriate transcription promoter and transcription terminator sequences. Plasmid pAYE640 was digested to completion with the restriction enzyme AflII and partially digested with the restriction enzyme HindIII and the 1.90 kb DNA fragment comprising the 3′ end of the yeast PRB1 promoter and the rHA coding sequence was isolated. Plasmid pDB2241 as described in patent application WO 00/44772, was digested with AflII/HindIII and the 4.31 kb DNA fragment comprising the 5′ end of the yeast PRB1 promoter and the yeast ADH1 terminator was isolated. The AflII/HindIII-DNA fragment from pAYE640 was them cloned into the AflII/HindIII pDB2241 vector DNA fragment to create the plasmid F. Plasmid F was digested to completion with PacI/XhoI and the 6.19 kb fragment isolated, the recessed ends were blunt ended with T4 DNA polymerase and dNTPs, and religated to generate plasmid G. Plasmid G was linearised with ClaI, the recessed ends were blunt ended with T4 DNA polymerase and dNTPs, and religated to generate plasmid H. Plasmid H was linearised with BlnI, the recessed ends were blunt ended with T4 DNA polymerase and dNTPs, and religated to generate plasmid I. Plasmid I was digested to completion with BmgBI/BglII, the 6.96 kg DNA fragment isolated and ligated to one of two double stranded oligonucleotide linkers, VC053 (SEQ ID NO:11)/VC054 (SEQ ID NO:12) and VC057 (SEQ ID NO:13)/VC058 (SEQ ID NO:14) to create plasmid J.

The recombinant albumin expression vector pDB2243 has been described previously in patent application WO 00/44772. Plasmid pDB2243 was digested to completion with SphI and Bsu36I and the 6.00 kb DNA fragment containing the yeast PRB1 promoter, most of a human albumin cDNA and part of the yeast ADH1 terminator was isolated. Plasmid J was digested to completion with SphI and AgeI and 1.93 kb DNA fragment containing the synthetic albumin gene and part of the yeast ADH1 terminator was isolated. A double stranded Bsu36I-AgeI DNA linker was formed by annealing two synthetic oligonucleotides having the sequences of SEQ ID NO:15 and SEQ ID NO:16.

Plasmid K, which contained an albumin dimer gene, was created by ligating the 6.00 kb SphI/Bsu36I DNA fragment from pDB2243, with the synthetic double stranded Bsu36I-AgeI DNA linker and the 1.93 kb SphI/AgeI DNA fragment from plasmid J.

Plasmid K was digested to completion with NotI and the NotI PRB1 promoter/albumin dimer gene/ADH1 terminator expression cassette is purified. The NotI albumin dimer expression cassette from plasmid K is ligated into NotI linearised pSAC35 (Sleep et al. (1991), Bio/technology 9: 183-187), which is previously treated with calf intestinal phosphatase (CIP) to create plasmid L. Plasmid L can be shown to contain the NotI expression cassette inserted into the NotI site of pSAC35 and orientated so that the expression of the fusion gene is away from the LEU2 auxotrophic marker and toward the 2 μm origin of replication. The sequence of the fusion gene in plasmid L is given as SEQ ID NO:17.

A yeast strain was transformed with plasmid L, the transformant fermented to produce an albumin dimer, and albumin dimer purified as described in Example 1 above.

1-32. (canceled)

33. An agent having a greater half-life than naturally produced albumin in a patient with nephrotic syndrome, the agent comprising an albumin-like first polypeptide bound to a second polypeptide, wherein the second polypeptide, when bound to the albumin-like first polypeptide, is therapeutically inert.

34. An agent according to claim 33 wherein the second polypeptide is an albumin-like polypeptide and wherein the two albumin-like polypeptides are covalently joined to one another other than solely by means of one or more cysteine-cysteine disulphide bridges.

35. A pharmaceutical composition comprising a pharmaceutically acceptable carrier or diluent and an agent having a greater half-life than naturally produced albumin in a patient with nephrotic syndrome, the agent comprising an albumin-like first polypeptide bound to a second polypeptide, wherein the second polypeptide, when bound to the albumin-like first polypeptide, is therapeutically inert.

36. A composition according to claim 35 wherein the second polypeptide is an albumin-like polypeptide and wherein more than 30% of the albumin-like first polypeptide present in the pharmaceutical composition is bound to the albumin-like second polypeptide.

37. An agent according to claim 33 wherein the agent is about 80-135 kDa in size.

38. A composition according to claim 35 wherein the agent is about 80-135 kDa in size.

39. An agent according to claim 33 wherein the second polypeptide is an albumin-like polypeptide or fragment thereof.

40. A composition according to claim 35 wherein the second polypeptide is an albumin-like polypeptide or fragment thereof.

41. An agent according to claim 33 wherein the albumin-like first polypeptide is bound to the second polypeptide via a peptide bond.

42. A composition according to claim 35 wherein the albumin-like first polypeptide is bound to the second polypeptide via a peptide bond.

43. An albumin ligand comprising an albumin-binding region that is capable of specifically binding to albumin wherein the ligand has a size of from 14 to 200 kDa.

44. An albumin ligand comprising an albumin-binding region that is capable of specifically binding to albumin wherein the ligand has a second binding site capable of binding specifically to a second polypeptide.

45. An albumin ligand according to claim 44 wherein the region that specifically binds to albumin is an antibody or a fragment there, such as Fab, Fv, ScFv or dAbs.

46. An albumin ligand according to claim 45 wherein the antibody is IgG.

47. An albumin ligand according to claim 44 further comprising a second polypeptide bound to the albumin-binding region.

48. An agent according to claim 33 wherein the agent is a fusion protein.

49. A composition according to claim 35 wherein the agent is a fusion protein.

50. An albumin ligand according to claim 44 wherein the ligand is a fusion protein.

51. An agent according to claim 33 wherein the bond between the albumin-like first polypeptide and the second polypeptide is not a peptide bond.

52. A composition according to claim 35 wherein the bond between the albumin-like first polypeptide and the second polypeptide is not a peptide bond.

53. A polynucleotide comprising a sequence that encodes a fusion protein as defined in claim 48.

54. A polynucleotide comprising a sequence that encodes a fusion protein as defined in claim 50.

55. A polynucleotide comprising a sequence that is suitable for introducing a non-natural epitope or a second polypeptide sequence into an albumin gene by homologous recombination.

56. A polynucleotide according to claim 53, and further comprising a regulatory region operatively linked to the coding sequence.

57. A polynucleotide according to claim 54, and further comprising a regulatory region operatively linked to the coding sequence.

58. A polynucleotide according to claim 55, and further comprising a regulatory region operatively linked to the coding sequence.

59. A vector comprising a polynucleotide as defined in claim 53.

60. A vector comprising a polynucleotide as defined in claim 54.

61. A vector comprising a polynucleotide as defined in claim 55.

62. A host cell comprising a polynucleotide according to claim 53.

63. A host cell comprising a polynucleotide according to claim 54.

64. A host cell comprising a polynucleotide according to claim 55.

65. A method for producing an agent as defined in claim 48 comprising the steps of growing a cell as defined in claim 62 and harvesting the agent produced by the cell.

66. A method for producing an albumin ligand as defined in claim 50 comprising the steps of growing a cell as defined in claim 63 and harvesting the albumin ligand produced by the cell.

67. A method according to claim 65 wherein the step of growing the cell comprises culturing the cell in a culture medium and the step of harvesting the agent comprises isolating the agent from the cell or from the medium.

68. A method according to claim 66 wherein the step of growing the cell comprises culturing the cell in a culture medium and the step of harvesting the albumin ligand comprises isolating the albumin ligand from the cell or from the medium.

69. A method for producing an agent having a greater half-life than naturally produced albumin in a patient with nephritic syndrome, agent comprising albumin-like first polypeptide bound to a second polypeptide, wherein the second polypeptide is therapeutically inert, comprising the steps of:
(a) providing, as a first component, an albumin-like first polypeptide;
(b) providing, as a second component, a second polypeptide; and
(c) contacting the first component with the second component under conditions suitable to allow the formation of the agent.

70. A method for producing an albumin ligand capable of binding to albumin to produce a molecule having a greater half-life than naturally produced albumin in a patient with nephritic syndrome, the albumin ligand comprising an albumin-binding region bound to a second polypeptide wherein the second polypeptide is therapeutically inert, comprising the steps of:
(a) providing, as a first component, a molecule comprising an albumin-binding region;
(b) providing, as a second component, the second polypeptide; and
(c) contacting the first component with the second component under conditions suitable to allow the formation of the albumin ligand.

71. A method for producing a composition comprising producing an agent by a method according to claim 69 and formulating the agent with a pharmaceutically acceptable carrier or diluent.

72. A method for producing a composition comprising producing an albumin ligand by a method according to claim 70 and formulating the agent with a pharmaceutically acceptable carrier or diluent.

73. Use of an agent comprising an agent as defined in claim 33 in the manufacture of a medicament for treating a renal disorder or a condition associated therewith.

74. Use of an albumin ligand comprising an albumin ligand as defined in claim 43 in the manufacture of a medicament for treating a renal disorder or a condition associated therewith.

75. Use of an albumin ligand comprising an albumin ligand as defined in claim 44 in the manufacture of a medicament for treating a renal disorder or a condition associated therewith.

76. A method for treating a patient with a renal disorder or a condition associated therewith comprising administering to the patient an agent as defined in claim 33 in a pharmaceutically effective amount and concentration.

77. A method for treating a patient with a renal disorder or a condition associated therewith comprising administering to the patient an albumin ligand as defined in claim 43 in a pharmaceutically effective amount and concentration.

78. A method for treating a patient with a renal disorder or a condition associated therewith comprising administering to the patient an albumin ligand as defined in claim 44 in a pharmaceutically effective amount and concentration.

79. A method according to claim 76 wherein the renal disorder is nephrotic syndrome or uremia.

80. A method according to claim 77 wherein the renal disorder is nephrotic syndrome or uremia.

81. A method according to claim 78 wherein the renal disorder is nephrotic syndrome or uremia.

82. A system comprising p1 (a) as a first component, a modified albumin comprising a non-natural epitope; and
(b) as a second component, an albumin ligand which binds specifically to the non-natural ligand of the modified albumin.

83. A system according to claim 82 wherein the first and second components are provided in the form of composition formulated with a pharmaceutical acceptable carrier or diluent.

84. A system according to claim 82 wherein the non-natural epitope is a C-terminal or N-terminal extension of the albumin molecule.

85. A system according to claim 82 for use in a method of treatment of the human or animal body by therapy or diagnosis.

86. Use of a system according to claim 82 in the manufacture of a medicament for the treatment of a renal disorder or a condition associated therewith.

87. A method for treating a patient with a renal disorder or condition associated therewith comprising administering to the patient the first and second components of a system as defined by claim 82, wherein the administration of the first and second components being simultaneous, separate or sequential.


SEQUENCE LISTING (17 sequences)

SEQ ID NO: 1 - DNA, 28 bp, Artificial Sequence
  5′ UTR from Saccharomyces cerevisiae
aagcttaacctaattctaacaagcaaag

SEQ ID NO: 2 - DNA, 72 bp, Artificial Sequence
  HSA/MFa1 Fusion Leader Sequence
atgaagtgggtttctttcatttccttgttgttcttgttctcctctgcttactctagatctttggataagaga

SEQ ID NO: 3 - DNA, 1755 bp, Artificial Sequence
  Codon optimised coding region for mature human albumin
gacgctcacaagtccgaagtcgctcacagattcaaggacttgggtgaagaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttg

SEQ ID NO: 4 - DNA, 10 bp, Artificial Sequence
  Translation Termination Site
taataagctt

SEQ ID NO: 5 - DNA, 3620 bp, Artificial Sequence
  Fusion Construct of Example 1
aagcttaacctaattctaacaagcaaagatgaagtgggtttctttcatttccttgttgttcttgttctcctctgcttactctagatctttggataagagagacgctcacaagtccgaagtcgctcacagattcaaggacttgggtgaagaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttggacgctcacaagtccgaagtcgctcacagattcaaggacttgggtgaagaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttgtaataagctt

SEQ ID NO: 6 - DNA, 36 bp, Artificial Sequence
  Oligonucleotide primer PRBJM1
gcatgcggccgcccgtaatgcggtatcgtgaaagcg

SEQ ID NO: 7 - DNA, 40 bp, Artificial Sequence
  Oligonucleotide primer PRBJM3
gttagaattaggttaagcttgtttttttattggcgatgaa

SEQ ID NO: 8 - DNA, 1827 bp, Artificial Sequence
  Genosys synthesised sequence of Example 2
atgaagtgggtttctttcatttccttgttgttcttgttctcctctgcttactctagatctttggataagagagacgctcacaagtccgaagtcgctcacagattcaaggacttgggtgaagaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttg

SEQ ID NO: 9 - DNA, 36 bp, Artificial Sequence; identical to SEQ ID NO: 6
  Oligonucleotide primer PRBJM1

SEQ ID NO: 10 - DNA, 40 bp, Artificial Sequence; identical to SEQ ID NO: 7
  Oligonucleotide primer PRBJM3

SEQ ID NO: 11 - DNA, 48 bp, Artificial Sequence
  Oligonucleotide linker VC053
gatctttggataagagagacgctcacaagtccgaagtcgctcaccggt

SEQ ID NO: 12 - DNA, 50 bp, Artificial Sequence
  Oligonucleotide linker VC054
ccttgaaccggtgagcgacttcggacttgtgagcgtctctcttatccaaa

SEQ ID NO: 13 - DNA, 86 bp, Artificial Sequence
  Oligonucleotide linker VC057
tcaaggacctaggtgaggaaaacttcaaggctttggtcttgatcgctttcgctcaatacttgcaacaatgtccattcgaagatcac

SEQ ID NO: 14 - DNA, 80 bp, Artificial Sequence
  Oligonucleotide linker VC058
gtgatcttcgaatggacattgttgcaagtattgagcgaaagcgatcaagaccaaagccttgaagttttcctcacctaggt

SEQ ID NO: 15 - DNA, 35 bp, Artificial Sequence
  Oligonucleotide linker Bsu36I-AgeI 1
ttaggcttagacgctcacaagtccgaagtcgctca

SEQ ID NO: 16 - DNA, 36 bp, Artificial Sequence
  Oligonucleotide linker Bsu36I-AgeI 2
ccggtgagcgacttcggacttgtgagcgtctaagcc

SEQ ID NO: 17 - DNA, 3620 bp, Artificial Sequence
  Fusion Construct of Example 2
aagctaaacctaattctaacaagcaaagatgaagtgggtaagctttatttcccttctttttctctttagctcggcttattccaggagcttggataaaagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttagacgctcacaagtccgaagtcgctcaccggttcaaggacctaggtgaggaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttgtaataagctt